(19) United States
(12) Patent Application Publication
(10) Pub. No.: US 2025/0255660 A1
(43) Pub. Date: Aug. 14, 2025

(54) CRYOABLATION CATHETER AND A CRYOABLATION CATHETER SYSTEM

(71) Applicant: KANEKA CORPORATION, Osaka (JP)

(72) Inventors: Hironori Takata, Osaka (JP); Shintaro Osumi, Osaka (JP)

(73) Assignee: KANEKA CORPORATION, Osaka (JP)

(21) Appl. No.: 19/193,737

(22) Filed: Apr. 29, 2025

Related U.S. Application Data
(63) Continuation of application No. PCT/JP2023/038306, filed on Oct. 24, 2023.

(30) Foreign Application Priority Data
Nov. 9, 2022 (JP) 2022-179429

(51) Int. Cl. A61B 18/02; A61B 18/00; A61M 25/00; A61M 25/01

(52) U.S. Cl. CPC A61B 18/02; A61M 25/003; A61M 25/0032; A61M 25/0068; A61B 2018/0022; A61B 2018/00494; A61B 2018/00577; A61B 2018/0212; A61M 2025/0004; A61M 25/0108; A61M 2205/0238; A61M 2210/1057

(57) ABSTRACT

A cryoablation catheter including an outer tube, an inner tube whose external shape is an irregular shape and a discharge flow path, is provided. The inner tube includes a guide wire lumen and a plurality of supply lumens. The discharge flow path is located between an inner surface of the outer tube and an outer surface of the inner tube, and allows the fluid to pass therethrough from a distal side to a proximal side of the outer tube. The inner tube includes a hole located in a distal portion of the inner tube. Any of the supply lumens and the discharge flow path are in communication with each other through the hole.

### TECHNICAL FIELD

One or more embodiments of the present invention relate to a cryoablation catheter and a cryoablation catheter system.

### BACKGROUND

Cryoablation techniques are medical techniques that involve bringing a device having been set to have a low temperature into contact with a target tissue so as to freeze and necrose cells forming the target tissue. The cryoablation techniques are used for therapies for cardiac muscle tissues and against tumor tissues. As methods for setting a device to have a low temperature, there are a method in which liquid nitrogen is used and a method in which the Joule-Thomson effect based on high-pressure gas is utilized.

Patent Document 1 describes a cryosurgical therapy catheter having a catheter body including a proximal end portion, a distal end portion, and a main lumen penetrating the proximal end portion and the distal end portion. A balloon for accommodating a low-temperature fluid supplied through the main lumen is placed over an orifice in the catheter body forming the cryosurgical therapy catheter, and the low-temperature fluid is supplied through the catheter body into the balloon. Consequently, the balloon is bulged, and the lesion is cooled.

### Patent Document

Patent Document 1: Japanese Translation of PCT International Application Publication No. JP-T-2001-524345

The catheter needs to be inserted into a duodenal papilla in order to freeze a luminal tissue located on the liver side, the gallbladder side, or the pancreas side relative to the duodenal papilla. For example, in order to insert the catheter into a bile duct via the duodenal papilla, the catheter needs to be significantly bent when being moved from the duodenum side to the bile duct side. The catheter for the cryoablation techniques described in Patent Document 1 is designed such that the area of contact between the fluid as a coolant and a wall surface that is located in the catheter and that defines the lumen is set to be as small as possible in order to make it less likely for the temperature of the coolant to increase before the coolant is carried to a target tissue. Accordingly, it is common to form only one lumen for supplying the fluid. Another purpose of forming only one lumen for supplying the fluid is to make it easy to decrease the diameter of the catheter in order to enable insertion into a narrower body cavity. However, such a conventional catheter in which only one lumen for supplying the fluid is formed is bent while passing through a bent body cavity. Consequently, the lumen for supplying the low-temperature fluid is closed, whereby the catheter cannot stably send the fluid.

### SUMMARY

One or more embodiments of the present invention have been made in view of the aforementioned circumstances, and a cryoablation catheter and a cryoablation catheter system, the diameters of which are easily decreased and which can stably send a fluid from a proximal side to a distal side, are provided.

One embodiment of a cryoablation catheter is as follows.
[1] A cryoablation catheter comprising:
an outer tube having a distal end and a proximal end and extending in a longitudinal direction;
an inner tube extending in the longitudinal direction and disposed in an inner cavity of the outer tube; and
a discharge flow path, wherein,
in a cross section perpendicular to the longitudinal direction, an external shape of the inner tube is an irregular shape,
in the cross section perpendicular to the longitudinal direction, the inner tube has a guide wire lumen into which a guide wire is inserted and a plurality of supply lumens which allow a fluid to pass therethrough from a proximal side to a distal side of the inner tube and which are formed in regions different from a region in which the guide wire lumen is formed,
the discharge flow path is located between an inner surface of the outer tube and an outer surface of the inner tube and allows the fluid to pass therethrough from a distal side to a proximal side of the outer tube, and
the inner tube has a distal portion in which a hole through which any of the supply lumens and the discharge flow path are in communication with each other is formed.

The fluid for cooling a lesion is carried from a proximal side to a distal side of the catheter through the supply lumens. The carried fluid is jetted from the hole to the outer side in the radial direction of the outer tube, and a lesion located on the outer side in the radial direction relative to the outer tube is cooled. The fluid that has come to have an increased temperature as a result of cooling the lesion is carried from the distal side to the proximal side of the catheter through the discharge flow path. In the cryoablation catheter according to one or more embodiments of the present invention, the external shape of the inner tube is an irregular shape, whereby the distance in the radial direction of the outer tube between the inner surface of the outer tube and the outer surface of the inner tube varies depending on the position. Consequently, even in a case where the distance between the inner surface of the outer tube and the outermost surface of the inner tube is shortened by decreasing the outer diameter of the outer tube, the fluid can move from the distal side to the proximal side by passing through positions at each of which the distance in the radial direction of the outer tube between the inner surface of the outer tube and the outer surface of the inner tube is comparatively long. Therefore, the passage capability of the cryoablation catheter according to one or more embodiments of the present invention can be improved by decreasing the diameter thereof, and the cryoablation catheter allows the fluid to stably move from the distal side to the proximal side. In addition, in the inner tube of the cryoablation catheter according to one or more embodiments of the present invention, the plurality of supply lumens are formed. Thus, even when one of the supply lumens is closed owing to bending of the cryoablation catheter which is passing through a bent body cavity, the fluid can be sent through the remaining supply lumens. Consequently, the fluid can be stably sent from the proximal side to the distal side. Furthermore, in the cryoablation catheter according to one or more embodiments of the present invention, the external shape of the inner tube is an irregular shape in the cross section perpendicular to the longitudinal direction of the outer tube, whereby stabilization of the position of the inner tube in the inner cavity of the outer tube can be facilitated.

Other aspects of the cryoablation catheter are as shown in the following [2] to [10]:
[2] The cryoablation catheter according to above [1], wherein, in the cross section perpendicular to the longitudinal direction, the external shape of the inner tube is a polygonal shape.
[3] The cryoablation catheter according to above [1], wherein, in the cross section perpendicular to the longitudinal direction, the external shape of the inner tube is a triangular shape.
[4] The cryoablation catheter according to any one of above [1] to [3], wherein,
in the cross section perpendicular to the longitudinal direction, the inner tube has a vertex portion protruding outward in a radial direction of the outer tube, and
the hole is formed in the vertex portion.
[5] The cryoablation catheter according to any one of above [1] to [4], wherein,
in the cross section perpendicular to the longitudinal direction, a roundness of an external shape of the outer tube obtained according to the following formula is higher than a roundness of the external shape of the inner tube obtained according to the following formula,

(Rmax−Rmin)/Rmin, where [Formula]

Rmax represents a radius of a circumscribed circle, and Rmin represents a radius of an inscribed circle.
[6] The cryoablation catheter according to any one of above [1] to [5], wherein, in the cross section perpendicular to the longitudinal direction, the guide wire lumen is formed at a position that overlaps with a centroid of the inner tube, and each of the supply lumens is formed at a position that does not overlap with the centroid of the inner tube.
[7] The cryoablation catheter according to any one of above [1] to [6], wherein, in the cross section perpendicular to the longitudinal direction, the plurality of supply lumens are located on an imaginary circle having a center at a centroid of the guide wire lumen.
[8] The cryoablation catheter according to any one of above [1] to [7], wherein, in the cross section perpendicular to the longitudinal direction, a maximum width of each of the supply lumens is smaller than a maximum width of the guide wire lumen.
[9] The cryoablation catheter according to any one of above [1] to [8], wherein the outer tube has, on a distal portion thereof, a balloon expandable and shrinkable in a radial direction of the outer tube.

[10] The cryoablation catheter according to any one of above [1] to [9], wherein
the cryoablation catheter further comprises a distal tip in which a lumen extending in the longitudinal direction is formed and which has an outer diameter decreasing from a proximal side toward a distal side, and
a distal end portion of the outer tube and a distal end portion of the inner tube are fixed to a proximal end portion of the distal tip.

One or more embodiments of the present invention also provide the following.
[11] A cryoablation catheter system comprising:
the cryoablation catheter according to any one of above [1] to [10]; and
a fluid supply device configured to supply the fluid to the supply lumens, wherein
the inner tube is connected to the fluid supply device.

In the cryoablation catheter according to one or more embodiments of the present invention, the external shape of the inner tube is an irregular shape, whereby the distance in the radial direction of the outer tube between the inner surface of the outer tube and the outer surface of the inner tube varies depending on the position. Consequently, even in a case where the distance between the inner surface of the outer tube and the outermost surface of the inner tube is shortened by decreasing the outer diameter of the outer tube, the fluid can move from the distal side to the proximal side by passing through positions at each of which the distance in the radial direction of the outer tube between the inner surface of the outer tube and the outer surface of the inner tube is comparatively long. Therefore, the passage capability of the cryoablation catheter according to one or more embodiments of the present invention can be improved by decreasing the diameter thereof, and the cryoablation catheter allows the fluid to stably move from the distal side to the proximal side. In addition, in the inner tube of the cryoablation catheter according to one or more embodiments of the present invention, the plurality of supply lumens are formed. Thus, even when one of the supply lumens is closed owing to bending of the cryoablation catheter which is passing through a bent body cavity, the fluid can be sent through the remaining supply lumens. Consequently, the fluid can be stably sent from the proximal side to the distal side. Furthermore, in the cryoablation catheter according to one or more embodiments of the present invention, the external shape of the inner tube is an irregular shape in the cross section perpendicular to the longitudinal direction of the outer tube, whereby stabilization of the position of the inner tube in the inner cavity of the outer tube can be facilitated. The cryoablation catheter system according to one or more embodiments of the present invention including the above cryoablation catheter also has the same advantageous effect.

### BRIEF DESCRIPTION OF THE DRAWINGS

FIG. 1 is a side view showing an example of the cryoablation catheter according to one or more embodiments of the present invention.

FIG. 2 is a cross-sectional view of the cryoablation catheter shown in FIG. 1.

FIG. 3 is a cross-sectional view, at the line III-III, of the cryoablation catheter shown in FIG. 2.

FIG. 4 is a cross-sectional view, at the line IV-IV, of the cryoablation catheter shown in FIG. 2.

FIG. 5 is a side view showing a modification of the cryoablation catheter according to one or more embodiments of the present invention.

FIG. 6 is a cross-sectional view of the cryoablation catheter shown in FIG. 5.

FIG. 7 is a cross-sectional view, at the line VII-VII, of the cryoablation catheter shown in FIG. 6.

FIG. 8 is a cross-sectional view, at the line VIII-VIII, of the cryoablation catheter shown in FIG. 6.

FIG. 9 is a cross-sectional view, at the line IX-IX, of the cryoablation catheter shown in FIG. 2.

FIG. 10 is a cross-sectional view showing a modification of the cryoablation catheter shown in FIG. 3.

### DETAILED DESCRIPTION

One or more embodiments of the present invention will be specifically explained below based on the following embodiments, however, the present invention is not restricted by the embodiments described below of course, and can be certainly put into practice after appropriate modifications within in a range meeting the gist of the above and the below, all of which are included in the technical scope of the present invention. In the drawings, hatching, a reference sign for a member may be omitted for convenience, and in such a case, the description and other drawings should be referred to. In addition, sizes of various members in the drawings may differ from the actual sizes thereof, since priority is given to understanding the features of one or more embodiments of the present invention.

A cryoablation catheter according to one or more embodiments of the present invention is a cryoablation catheter having the following gist. That is, the cryoablation catheter includes: an outer tube having a distal end and a proximal end and extending in a longitudinal direction; an inner tube extending in the longitudinal direction of the outer tube and disposed in an inner cavity of the outer tube; and a discharge flow path. In a cross section perpendicular to the longitudinal direction of the outer tube, an external shape of the inner tube is an irregular shape. In the cross section perpendicular to the longitudinal direction of the outer tube, the inner tube has a guide wire lumen into which a guide wire is inserted and a plurality of supply lumens which allow a fluid to pass therethrough from a proximal side to a distal side of the inner tube and which are formed in regions different from a region in which the guide wire lumen is formed. The discharge flow path is located between an inner surface of the outer tube and an outer surface of the inner tube and allows the fluid to pass therethrough from a distal side to a proximal side of the outer tube. The inner tube has a distal portion in which a hole through which any of the supply lumens and the discharge flow path are in communication with each other is formed.

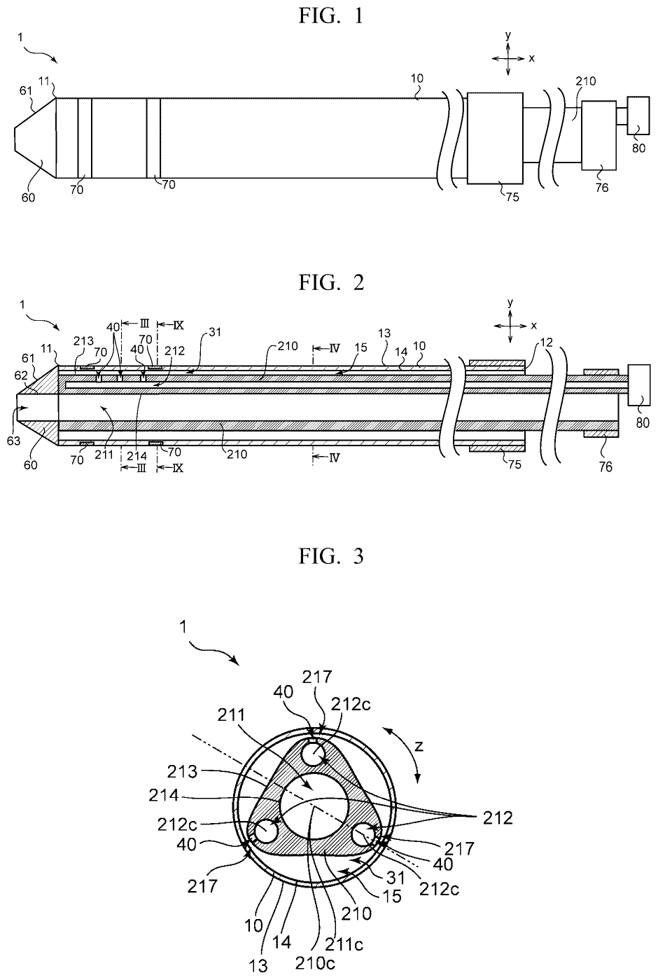

An overall configuration of a cryoablation catheter 1 according to one or more embodiments of the present invention will be described with reference to FIG. 1 to FIG. 10. The cryoablation catheter 1 shown in FIG. 1 to FIG. 10 includes an outer tube 10 and an inner tube 210. In these drawings, the longitudinal direction and the radial direction of the outer tube 10 are respectively indicated by x and y. The radial direction y is a direction perpendicular to the longitudinal direction x. In addition, the circumferential direction of the outer tube 10 is indicated by z. Hereinafter, the cryoablation catheter 1 is sometimes simply referred to as the catheter 1.

In the present description, the proximal side refers to the hand side of a user in an extension direction of the outer tube, and the distal side refers to the side opposite to the proximal side, i.e., a treatment target side. A distal portion of each member refers to the distal half of the member, and a proximal portion of each member refers to the proximal half of the member.

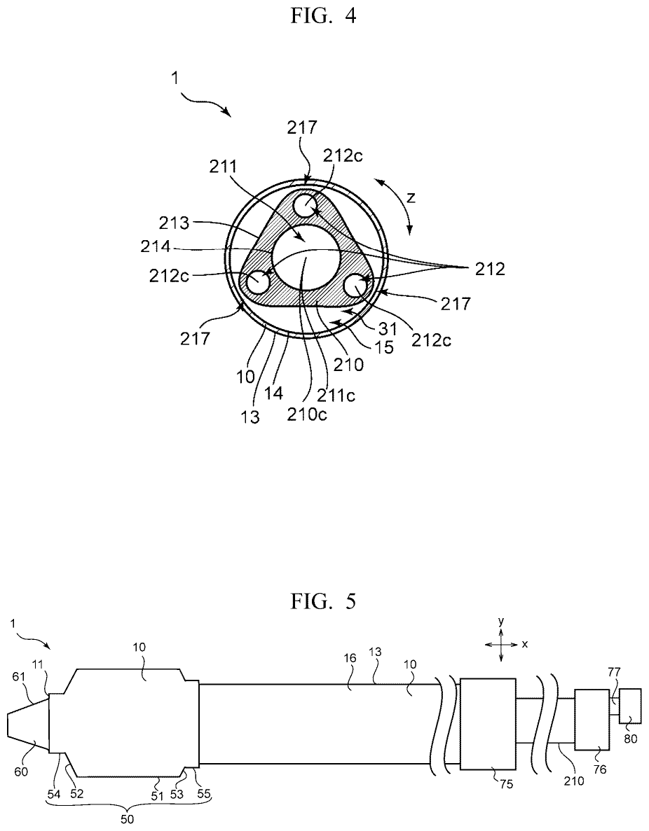

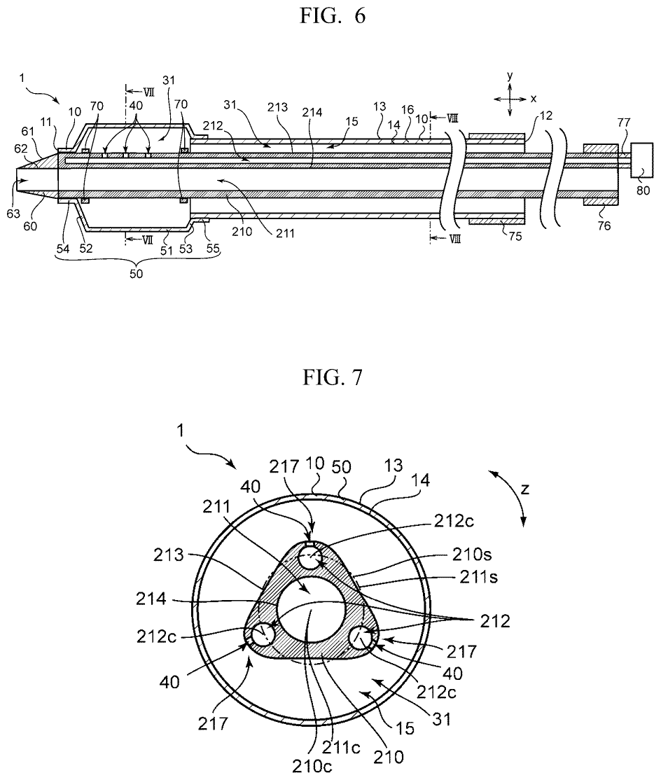

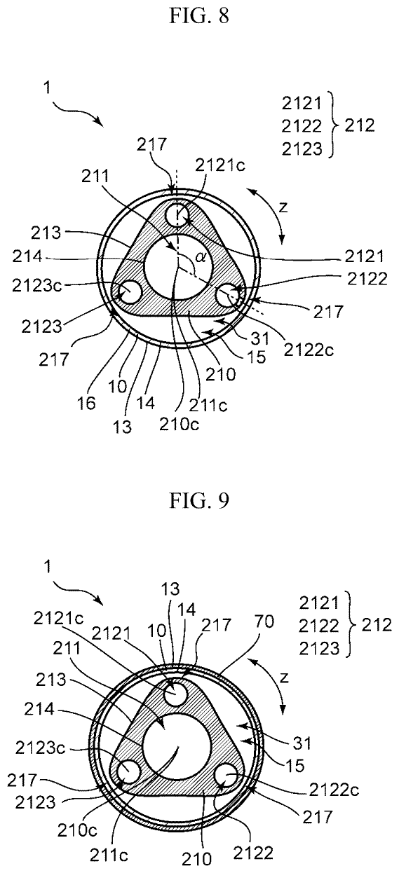

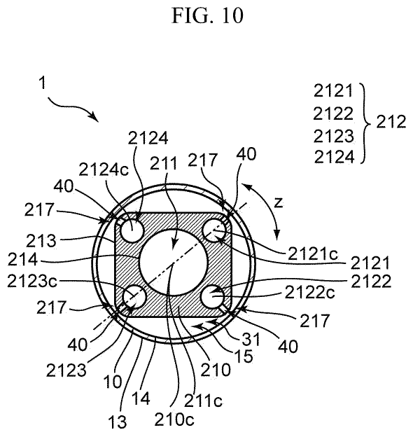

FIG. 1 is a side view showing an example of the cryoablation catheter according to one or more embodiments of the present invention. FIG. 2 is a cross-sectional view of the cryoablation catheter shown in FIG. 1. FIG. 3 is a cross-sectional view, at the line III-III, of the cryoablation catheter shown in FIG. 2. FIG. 4 is a cross-sectional view, at the line IV-IV, of the cryoablation catheter shown in FIG. 2. FIG. 5 is a side view showing a modification of the cryoablation catheter according to one or more embodiments of the present invention. FIG. 6 is a cross-sectional view of the cryoablation catheter shown in FIG. 5. FIG. 7 is a cross-sectional view, at the line VII-VII, of the cryoablation catheter shown in FIG. 6. FIG. 8 is a cross-sectional view, at the line VIII-VIII, of the cryoablation catheter shown in FIG. 6. FIG. 9 is a cross-sectional view, at the line IX-IX, of the cryoablation catheter shown in FIG. 2. FIG. 10 is a cross-sectional view showing a modification of the cryoablation catheter shown in FIG. 3.

As shown in FIG. 1 to FIG. 10, the outer tube 10 provided to the catheter 1 has a distal end 11 and a proximal end 12 and extends in the longitudinal direction x. The outer tube 10 has an inner cavity 15. The inner cavity 15 may extend in the longitudinal direction x of the outer tube 10. The outer tube 10 has an outer surface 13 facing the outside of the outer tube 10 and an inner surface 14 facing the inner cavity 15 of the outer tube 10.

As shown in FIG. 1 to FIG. 10, the inner tube 210 provided to the catheter 1 extends in the longitudinal direction x of the outer tube 10 and is disposed in the inner cavity 15 of the outer tube 10. As shown in FIG. 3, FIG. 4, FIG. 7 and FIG. 8, the inner tube 210 has, in the cross section perpendicular to the longitudinal direction x of the outer tube 10, a guide wire lumen 211 into which a guide wire is inserted and a plurality of supply lumens 212 which allow a fluid to pass therethrough from the proximal side to the distal side of the inner tube 210 and which are formed in regions different from a region in which the guide wire lumen 211 is formed.

As shown in FIG. 3 and FIG. 10, in the cross section perpendicular to the longitudinal direction x of the outer tube 10, the external shape of the inner tube 210 is an irregular shape. The irregular shape means a shape other than perfect circular shapes, and the perfect circular shapes do not include elliptical shapes or oval shapes. In a case where, for example, the term “circular shape” is used in the present description, the circular shape encompasses shapes such as perfect circular shapes, elliptical shapes, and oval shapes.

As shown in FIG. 2 to FIG. 4 and FIG. 6 to FIG. 10, the catheter 1 has a discharge flow path 31 which is located between the inner surface 14 of the outer tube 10 and an outer surface 213 of the inner tube 210 and which allows the fluid to pass therethrough from the distal side to the proximal side of the outer tube 10. The discharge flow path 31 is a space provided between the inner surface 14 of the outer tube 10 and the outer surface 213 of the inner tube 210. The fluid having passed through the supply lumens 212 so as to be carried from the proximal side to the distal side of the catheter 1 is discharged to the outside of the catheter 1 via the discharge flow path 31 which is a space provided between the inner surface 14 of the outer tube 10 and the outer surface 213 of the inner tube 210.

As shown in FIG. 2, FIG. 3, FIG. 6, FIG. 7, and FIG. 10, the inner tube 210 has a distal portion in which holes 40 through which the respective supply lumens 212 and the discharge flow path 31 are in communication with each other are formed. The fluid having passed through the supply lumens 212 so as to be carried from the proximal side to the distal side of the catheter 1 is jetted to the discharge flow path 31 via the holes 40.

The fluid passes through the supply lumens 212 so as to be carried from the proximal side to the distal side of the catheter 1. Then, the fluid is jetted from the holes 40 of the inner tube 210 to the outer side in the radial direction of the outer tube 10 and passes through the discharge flow path 31 so as to be carried from the distal side to the proximal side. Then, the fluid is discharged to the outside of the catheter 1.

In the catheter 1 according to one or more embodiments of the present invention, the external shape of the inner tube 210 is an irregular shape, whereby the distance in the radial direction y of the outer tube 10 between the inner surface 14 of the outer tube 10 and the outer surface 213 of the inner tube 210 varies depending on the position. Consequently, even in a case where the distance between the inner surface 14 of the outer tube 10 and the outermost surface of the inner tube 210 is shortened by decreasing the outer diameter of the outer tube 10, the fluid can move from the distal side to the proximal side by passing through positions at each of which the distance in the radial direction y of the outer tube 10 between the inner surface 14 of the outer tube 10 and the outer surface 213 of the inner tube 210 is comparatively long. Therefore, the passage capability of the catheter 1 according to one or more embodiments of the present invention can be improved by decreasing the diameter thereof, and the catheter 1 allows the fluid to stably move from the distal side to the proximal side. In addition, in the inner tube 210 of the catheter 1 according to one or more embodiments of the present invention, the plurality of supply lumens 212 are formed. Thus, even when one of the supply lumens 212 is closed owing to bending of the cryoablation catheter 1 which is passing through a bent body cavity, the fluid can be sent through the remaining supply lumens 212. Consequently, the fluid can be stably sent from the proximal side to the distal side. Furthermore, in the catheter 1 according to one or more embodiments of the present invention, the external shape of the inner tube 210 is an irregular shape in the cross section perpendicular to the longitudinal direction x of the outer tube 10, whereby stabilization of the position of the inner tube 210 in the inner cavity 15 of the outer tube 10 can be facilitated.

The catheter 1 may be a cryoablation catheter for duodenal papilla insertion. The cryoablation catheter for duodenal papilla insertion means a cryoablation catheter to be inserted into a duodenal papilla in order to freeze a luminal tissue that leads to the opening of the duodenal papilla and that is located on the liver side, the gallbladder side, or the pancreas side relative to the duodenal papilla.

The outer tube 10 may have flexibility so as to be inserted into a body. Consequently, the outer tube 10 can be deformed according to the shape of the body cavity. In addition, the outer tube 10 may have elasticity in order to retain the shape thereof.

Examples of the outer tube 10 include: a hollow member formed by arranging one or more wire materials in a predetermined pattern; a member formed by coating at least one of an inner surface or an outer surface of the above hollow member with a resin; a resin tube; and a member formed by a combination thereof, e.g., a member formed by connecting these outer tubes in a longitudinal axis direction. The hollow member formed by arranging a wire material in a predetermined pattern is exemplified by: a tubular member having a network structure as a result of simple intersection or weaving of the wire material; and a coil formed by winding the wire material. The wire material may be one or more single wires or may be one or more twisted wires. The resin tube may be produced through, for example, extrusion molding. In a case where the outer tube 10 is the resin tube, the outer tube 10 may be composed of a single layer or a plurality of layers. The outer tube 10 may be such that: one portion thereof extending in the longitudinal direction x or the circumferential direction z is composed of a single layer; and another portion thereof extending in the longitudinal direction x or the circumferential direction z is composed of a plurality of layers.

The outer tube 10 may be formed from, for example: a synthetic resin such as a polyolefin resin (e.g., polyethylene or polypropylene), a polyamide resin (e.g., nylon), a polyester resin (e.g., PET), an aromatic polyether ketone resin (e.g., PEEK), a polyether polyamide resin, a polyurethane resin, a polyimide resin, or a fluorine resin (e.g., PTFE, PFA, or ETFE); or a metal such as stainless steel, carbon steel, or a nickel-titanium alloy. These types of materials may be used singly, or two or more of these types of materials may be used in combination.

The shape of the outer tube 10 is a tubular shape and may be a shape such as the shape of a hollow column or a hollow polygonal prism.

Specifically, in the cross section perpendicular to the longitudinal direction x of the outer tube 10, the external shape of the outer tube 10 may be any of circular shapes, polygonal shapes including triangular shapes and quadrangular shapes, semi-circular shapes, fan shapes, wedge shapes, protruding shapes, a combination of these shapes, and the like, but may differ from the external shape of the inner tube 210. Since the inner tube 210 has an irregular shape other than perfect circular shapes, the external shape of the outer tube 10 may be a circular shape or a perfect circular shape, in the cross section perpendicular to the longitudinal direction x of the outer tube 10. By employing the above configuration, the distance in the radial direction y of the outer tube 10 between the inner surface 14 of the outer tube 10 and the outer surface 213 of the inner tube 210 varies depending on the position. Consequently, even in a case where the distance between the inner surface 14 of the outer tube 10 and the outermost surface of the inner tube 210 is shortened by decreasing the outer diameter of the outer tube 10, the fluid can move from the distal side to the proximal side by passing through positions at each of which the distance in the radial direction y of the outer tube 10 between the inner surface 14 of the outer tube 10 and the outer surface 213 of the inner tube 210 is comparatively long. Although not shown, the external shape of the outer tube 10 and the external shape of the inner tube 210 may be congruent with each other in the cross section perpendicular to the longitudinal direction x of the outer tube 10. For example, in a case where the external shape of the outer tube 10 and the external shape of the inner tube 210 are triangular shapes congruent with each other, each vertex portion of one of the triangular shapes and each vertex portion of the other triangular shape are located at different positions in the circumferential direction z of the outer tube 10. By doing so, the distance in the radial direction y of the outer tube 10 between the inner surface 14 of the outer tube 10 and the outer surface 213 of the inner tube 210 varies depending on the position, whereby the above advantageous effects can be obtained.

As shown in FIG. 1, FIG. 2, FIG. 6, and FIG. 9, radiopaque markers 70 may be provided on the distal portion of the outer tube 10. In this configuration, use of an X-ray imaging device makes it possible to visually recognize the position of the distal portion of the catheter 1. As shown in FIG. 1, FIG. 2, and FIG. 9, each of the radiopaque markers 70 may be provided on, for example, the outer surface 13 of the outer tube 10.

As shown in FIG. 6, the radiopaque markers 70 may be provided on the distal portion of the inner tube 210. In this configuration, use of an X-ray imaging device makes it possible to visually recognize the position of the distal portion of the catheter 1.

The shape of each of the above radiopaque markers 70 may be a tubular shape as shown in FIG. 1, FIG. 2, and FIG. 9. Other examples of the shape include the shape of a hollow column, the shape of a hollow polygonal prism, a shape obtained by forming a slit in a tube so as to have a C-shaped cross section, the shape of a coil obtained by winding a wire material, and the like.

As a material forming the above radiopaque marker 70, a radiopaque substance such as lead, barium, iodine, tungsten, gold, platinum, iridium, stainless steel, titanium, or a cobalt-chromium alloy may be used, for example. The radiopaque marker 70 may be obtained in such a manner that radiopaque particles made from barium sulfate or the like are dispersed in the outer tube 10, the inner tube 210, or a separately provided resin member.

As shown in FIG. 5 to FIG. 7, the outer tube 10 may have, on the distal portion thereof, a balloon 50 expandable and shrinkable in the radial direction y of the outer tube 10. FIG. 5 to FIG. 7 show states where the diameter of the balloon 50 is increased. The balloon 50 may be configured such that the diameter thereof is increased by supplying the fluid into the balloon 50 and is decreased by removing the fluid.

In a case where the outer tube 10 has the balloon 50, a portion of the outer tube 10 formed as the balloon 50 and a portion of the outer tube 10 other than the balloon 50 may be different components and may compose the outer tube 10 by being connected to each other. For example, as shown in FIG. 5 and FIG. 6, the outer tube 10 may be configured to have the balloon 50 and a tubular member 16. Alternatively, in a case where the outer tube 10 has the balloon 50, the portion of the outer tube 10 formed as the balloon 50 and the portion of the outer tube 10 other than the balloon 50 may be molded to be integrated with each other.

In the case where the catheter 1 has the balloon 50, the fluid passes through the supply lumens 212 so as to be carried from the proximal side to the distal side of the catheter 1 and is jetted from the holes 40 of the inner tube 210 to the outer side in the radial direction of the outer tube 10 so as to increase the diameter of the balloon 50. Then, the fluid passes through the discharge flow path 31 so as to be carried from the distal side to the proximal side and is discharged to the outside of the catheter 1. When the diameter of the balloon 50 is increased, the outer surface of the balloon 50 comes into contact with a biological tube wall of a blood vessel, a gastrointestinal tract, or the like, whereby the position of the catheter 1 in the body cavity can be stabilized. In addition, since the outer surface of the balloon 50 comes into contact with a biological tube wall of a blood vessel, a gastrointestinal tract, or the like, local freezing of a tissue with which the outer surface of the balloon 50 is in contact can be facilitated.

The diameter of the balloon 50 is increased by the fluid jetted from the holes 40 to the outer side in the radial direction of the outer tube 10 and is decreased by discharging the fluid. As shown in FIG. 5 and FIG. 6, in a state where the diameter of the balloon 50 is increased, the balloon 50 may have: a straight tube portion 51 having a substantially cylindrical shape; a distal-side tapered portion 52 located on the distal side relative to the straight tube portion 51 and having an outer diameter decreasing toward the distal side; and a proximal-side tapered portion 53 located on the proximal side relative to the straight tube portion 51 and having an outer diameter decreasing toward the proximal side. Furthermore, the balloon 50 may have: a distal-side sleeve portion 54 located on the distal side relative to the distal-side tapered portion 52 and fixed to the outer surface 213 of the inner tube 210, the distal-side sleeve portion 54 having a diameter that is not increased by the fluid jetted from the holes 40 to the outer side in the radial direction of the outer tube 10; and a proximal-side sleeve portion 55 located on the proximal side relative to the proximal-side tapered portion 53 and fixed to the outer surface of the tubular member 16, the proximal-side sleeve portion 55 having a diameter that is not increased by the fluid jetted from the holes 40 to the outer side in the radial direction of the outer tube 10.

Although a material forming the portion of the outer tube 10 formed as the balloon 50 and a material forming the portion of the outer tube 10 other than the balloon 50 may be identical to or different from each other, the material forming the balloon 50 may have a higher thermal conductivity than the material forming the portion of the outer tube 10 other than the balloon 50. Consequently, the temperature of the portion of the outer tube 10 formed as the balloon 50 is more easily decreased than the temperature of the portion of the outer tube 10 other than the balloon 50, whereby the tissue freezing efficiency at the location at which the balloon 50 is disposed is easily increased.

As shown in FIG. 6, the holes 40 may be located inside the balloon 50. That is, the holes 40 may be formed in portions of the inner tube 210 that are located inside the balloon 50. Although the holes 40 may be formed also in portions of the inner tube 210 that are not located inside the balloon 50, the holes 40 may be formed only in the portions of the inner tube 210 that are located inside the balloon 50. Consequently, the temperature of the portion of the outer tube 10 formed as the balloon 50 easily becomes lower than the temperature of the portion of the outer tube 10 other than the balloon 50, whereby the tissue freezing efficiency at the location at which the balloon 50 is disposed is easily increased.

As shown in FIG. 2 and FIG. 6, it may be preferable that, in the longitudinal direction x of the outer tube 10, a plurality of the radiopaque markers 70 are provided, one of the radiopaque markers 70 is located on the distal side relative to the holes 40, and another one of the radiopaque markers 70 is located on the proximal side relative to the holes 40. Consequently, use of an X-ray imaging device makes it possible to facilitate recognition of the positions of the holes 40.

As shown in FIG. 6, the radiopaque markers 70 may be provided on the outer surface 213 of the inner tube 210 so as to be located at positions corresponding, in the longitudinal direction x of the outer tube 10, to the positions of the distal end and the proximal end of the straight tube portion 51 of the balloon 50. Alternatively, although not shown in FIG. 6, the radiopaque markers 70 may each be provided on the outer surface 213 of the inner tube 210 so as to be located at a position corresponding, in the longitudinal direction x of the outer tube 10, to the position of the center of the straight tube portion 51 of the balloon 50. In this configuration, use of an X-ray imaging device makes it possible to visually recognize the position of the straight tube portion 51 of the balloon 50.

The inner tube 210 has the guide wire lumen 211 and the supply lumens 212. The guide wire lumen 211 and the supply lumens 212 may extend in the longitudinal direction x of the outer tube 10. The inner tube 210 has: the outer surface 213 facing the outside of the inner tube 210, i.e., facing the outer tube 10 side; and an inner surface 214 facing the guide wire lumen 211. As shown in FIG. 2, the guide wire lumen 211 may be in communication with a lumen 63 of a distal tip 60 described later.

As a material forming the inner tube 210, a synthetic resin, a metal, or the like which are the same as those for the outer tube 10 may be used. The material forming the inner tube 210 and the material forming the outer tube 10 may be identical to or different from each other.

The inner tube 210 may be molded as one piece from a predetermined material. The predetermined material includes a material obtained by mixing a plurality of substances or a material formed from only a single substance. The inner tube 210 can be molded as one piece through, for example, extrusion molding by using the predetermined material. By molding the inner tube 210 as one piece from the predetermined material, there is no seam between components, whereby portions having high and low rigidities are less likely to be generated. Consequently, it is possible to facilitate, when passing through a portion having a large bending angle such as a duodenal papilla, suppression of bending of the inner tube 210 and suppression of closure of the supply lumens 212. In addition, by molding the inner tube 210 as one piece from the predetermined material, it is unnecessary to manufacture a plurality of components, whereby a manufacturing process for the inner tube 210 can be simplified, and the time and cost required for the manufacturing can be decreased.

As shown in FIG. 1, FIG. 2, FIG. 5 and FIG. 6, the inner tube 210 may extend over the entire length of the outer tube 10. The catheter 1 shown in FIG. 1, FIG. 2, FIG. 5 and FIG. 6 is of a so-called over-the-wire type.

As described above, in the cross section perpendicular to the longitudinal direction x of the outer tube 10, the external shape of the inner tube 210 is an irregular shape. Specifically, the external shape of the inner tube 210 may be any of polygonal shapes including triangular shapes and quadrangular shapes, semi-circular shapes, fan shapes, wedge shapes, protruding shapes, a combination of these shapes, and the like. However, in the cross section perpendicular to the longitudinal direction x of the outer tube 10, the external shape of the inner tube 210 may be a polygonal shape as shown in FIG. 3 and FIG. 10 or a triangular shape as shown in FIG. 3. The polygonal shapes including triangular shapes and quadrangular shapes not only include polygonal shapes having definite vertices and having straight sides but also include: so-called rounded-corner polygonal shapes such as rounded-corner triangular shapes and rounded-corner quadrangular shapes having rounded corners; and polygonal shapes having sides that are at least partially curved. Alternatively, the external shape of the inner tube 210 may be an indefinite shape having a recess/protrusion, a chip, or the like.

As shown in FIG. 3, FIG. 7, and FIG. 10, it may be preferable that, in the cross section perpendicular to the longitudinal direction x of the outer tube 10, the inner tube 210 has vertex portions 217 protruding outward in the radial direction of the outer tube 10, and the holes 40 are formed in the vertex portions 217. Each of the vertex portions 217 is a portion protruding outward in the radial direction of the outer tube 10 in the cross section perpendicular to the longitudinal direction x of the outer tube 10. For example, a vertex portion of any of polygonal shapes including triangular shapes and quadrangular shapes, a rounded-corner portion of any of rounded-corner polygonal shapes such as rounded-corner triangular shapes and rounded-corner quadrangular shapes, and the like correspond to each of the vertex portions 217. Thus, the distance between the corresponding hole 40 and the inner surface 14 of the outer tube 10 can be shortened. Consequently, it is possible to suppress increase, in the temperature of the fluid jetted from the hole 40, that occurs before said fluid reaches the inner surface 14 of the outer tube 10. Therefore, the lesion can be efficiently cooled. Although not shown, the hole 40 may be formed in a portion of the inner tube 210 other than the vertex portion 217 in the cross section perpendicular to the longitudinal direction x of the outer tube 10. For example, in a case where the external shape of the inner tube 210 in the cross section perpendicular to the longitudinal direction x of the outer tube 10 is a triangular shape, the holes 40 may be formed at portions other than the vertices.

As shown in FIG. 3, it may be preferable that, in the cross section perpendicular to the longitudinal direction x of the outer tube 10, an eccentric distance from a centroid 210c of the inner tube 210 to a centroid 212c of each of the supply lumens 212 is longer than an eccentric distance from the centroid 210c of the inner tube 210 to a centroid 211c of the guide wire lumen 211. The eccentric distance from the centroid 210c of the inner tube 210 to the centroid 212c of each of the supply lumens 212 refers to the distance to the centroid 212c of the supply lumen 212 with the centroid 210c of the inner tube 210 being regarded as the center. The eccentric distance from the centroid 210c of the inner tube 210 to the centroid 211c of the guide wire lumen 211 refers to the distance to the centroid 211c of the guide wire lumen 211 with the centroid 210c of the inner tube 210 being regarded as the center. By setting the eccentric distance from the centroid 210c of the inner tube 210 to the centroid 212c of each of the supply lumens 212 to be longer than the eccentric distance from the centroid 210c of the inner tube 210 to the centroid 211c of the guide wire lumen 211, the supply lumen 212 is formed on the outer side in the radial direction of the inner tube 210 relative to the guide wire lumen 211. Consequently, the fluid carried to the distal side through the supply lumens 212 can be jetted via the holes 40 to the discharge flow path 31 comparatively early, whereby efficient cooling of a tissue as a therapy target can be facilitated.

As shown in FIG. 3, it may be preferable that, in the cross section perpendicular to the longitudinal direction x of the outer tube 10, the guide wire lumen 211 is formed at a position that overlaps with the centroid 210c of the inner tube 210, and each of the supply lumens 212 is formed at a position that does not overlap with the centroid 210c of the inner tube 210. By forming the guide wire lumen 211 at a position that overlaps with the centroid 210c of the inner tube 210 and by forming each of the supply lumens 212 at a position that does not overlap with the centroid 210c of the inner tube 210, the supply lumen 212 is formed on the outer side in the radial direction of the inner tube 210 relative to the guide wire lumen 211. Consequently, the fluid carried to the distal side through the supply lumens 212 can be jetted via the holes 40 to the discharge flow path 31 comparatively early, whereby efficient cooling of a tissue as a therapy target can be facilitated. In addition, by forming the guide wire lumen 211 at a position that overlaps with the centroid 210c of the inner tube 210, the guide wire is easily located at the axis of the inner tube 210, whereby improvement of the operability of the catheter 1 can be facilitated.

As shown in FIG. 3, it may be preferable that, in the cross section perpendicular to the longitudinal direction x of the outer tube 10, a roundness of the external shape of the outer tube 10 obtained according to the following formula is higher than a roundness of the external shape of the inner tube 210 obtained according to the following formula. In the present description, each roundness is obtained according to the following formula.

(Rmax−Rmin)/Rmin, where [Formula]

Rmax represents a radius of a circumscribed circle, and Rmin represents a radius of an inscribed circle.

By the above configuration, the distance in the radial direction of the outer tube 10 between the inner surface 14 of the outer tube 10 and the outer surface 213 of the inner tube 210 varies depending on the position. Consequently, even in a case where the distance between the inner surface 14 of the outer tube 10 and the outermost surface of the inner tube 210 is shortened by decreasing the outer diameter of the outer tube 10, the fluid can move from the distal side to the proximal side by passing through positions at each of which the distance in the radial direction of the outer tube 10 between the inner surface 14 of the outer tube 10 and the outer surface 213 of the inner tube 210 is comparatively long. Therefore, the passage capability of the catheter 1 according to one or more embodiments of the present invention can be improved by decreasing the diameter thereof, and the catheter 1 allows the fluid to stably move from the distal side to the proximal side. In addition, employment of the above configuration makes it possible to facilitate stabilization of the position of the inner tube 210 in the inner cavity 15 of the outer tube 10.

The shape of each of the holes 40 may be set to a shape such as a circular shape, an oval shape, or a polygonal shape. The number of the formed holes 40 may be one or may be two or more. All of a plurality of the holes 40 may have the same shape, or the holes 40 may have mutually different shapes.

As shown in FIG. 3, it may be preferable that, in the cross section perpendicular to the longitudinal direction x of the outer tube 10, one of the holes 40 is formed in one of the supply lumens 212. Although not shown, it is also allowed to employ a configuration in which, in the cross section perpendicular to the longitudinal direction x of the outer tube 10, a plurality of the holes 40 are formed in one of the supply lumens 212.

As shown in FIG. 3, it may be preferable that: a plurality of the holes 40 are formed; and, in the cross section perpendicular to the longitudinal direction x of the outer tube 10, only one of the holes 40 is present on the one straight line passing through the centroid 210c of the inner tube 210. In FIG. 3, the one straight line passing through the centroid 210c of the inner tube 210 is indicated by an alternate long and two short dashes line. When the catheter 1 is bent, a force is applied particularly to a portion that is located on the outer peripheral edge of the inner tube 210 and that intersects the one straight line passing through the centroid 210c of the inner tube 210. There are two such portions that are located on the outer peripheral edge of the inner tube 210 and that intersect the one straight line passing through the centroid 210c of the inner tube 210. One of the two portions receives a force that causes elongation in the longitudinal direction x of the outer tube 10, and the other one of the two portions receives a force that causes contraction in the longitudinal direction x of the outer tube 10. In particular, the portion in which each of the holes 40 is formed tends to have a lower rigidity and tends to be more easily bent than the other portion. Employment of a configuration in which, in the cross section perpendicular to the longitudinal direction x of the outer tube 10, only one of the holes 40 is present on the one straight line passing through the centroid 210c of the inner tube 210 makes it possible to facilitate retention of the rigidity of each of the portions that are located on the outer peripheral edge of the inner tube 210 and that intersect the one straight line passing through the centroid 210c of the inner tube 210. Thus, improvement of the resistance to bending can be facilitated. Consequently, suppression of closure of the supply lumens 212 due to bending of the catheter 1 can be facilitated. Alternatively, as shown in FIG. 10, it is also allowed to employ a configuration in which, in the cross section perpendicular to the longitudinal direction x of the outer tube 10, two of the holes 40 are present on the one straight line passing through the centroid 210c of the inner tube 210. In FIG. 10, the one straight line passing through the centroid 210c of the inner tube 210 is indicated by an alternate long and two short dashes line.

As shown in FIG. 3, it may be preferable that, in the cross section perpendicular to the longitudinal direction x of the outer tube 10, only one of the supply lumens 212 is present on one straight line passing through the centroid 210c of the inner tube 210 and any of the holes 40. In FIG. 3, the one straight line passing through the centroid 210c of the inner tube 210 and the hole 40 is indicated by the alternate long and two short dashes line. When the catheter 1 is bent, a force is applied particularly to a portion that is located on the outer peripheral edge of the inner tube 210 and that intersects the one straight line passing through the centroid 210c of the inner tube 210. There are two such portions that are located on the outer peripheral edge of the inner tube 210 and that intersect the one straight line passing through the centroid 210c of the inner tube 210. One of the two portions receives a force that causes elongation in the longitudinal direction x of the outer tube 10, and the other one of the two portions receives a force that causes contraction in the longitudinal direction x of the outer tube 10. In particular, the portion in which each of the holes 40 is formed tends to have a lower rigidity and tends to be more easily bent than the other portion. Employment of a configuration in which, in the cross section perpendicular to the longitudinal direction x of the outer tube 10, only one of the supply lumens 212 is present on the one straight line passing through the centroid 210c of the inner tube 210 and the hole 40 makes it possible to facilitate retention of the rigidity of each of the portions that are located on the outer peripheral edge of the inner tube 210 and that intersect the one straight line passing through the centroid 210c of the inner tube 210. Thus, improvement of the resistance to bending can be facilitated. Consequently, suppression of closure of the supply lumens 212 due to bending of the catheter 1 can be facilitated. Alternatively, as shown in FIG. 10, it is also allowed to employ a configuration in which, in the cross section perpendicular to the longitudinal direction x of the outer tube 10, two of the supply lumens 212 are present on the one straight line passing through the centroid 210c of the inner tube 210 and the hole 40. In FIG. 10, the one straight line passing through the centroid 210c of the inner tube 210 and the hole 40 is indicated by the alternate long and two short dashes line.

As shown in FIG. 7, it may be preferable that, in the cross section perpendicular to the longitudinal direction x of the outer tube 10, the plurality of supply lumens 212 are located on an imaginary circle 210s having a center at the centroid 210c of the inner tube 210. In FIG. 7, one such imaginary circle 210s having a center at the centroid 210c of the inner tube 210 is indicated by an alternate long and two short dashes line. It may be preferable that, in the cross section perpendicular to the longitudinal direction x of the outer tube 10, all of the plurality of supply lumens 212 formed in the inner tube 210 are located on the one imaginary circle 210s having a center at the centroid 210c of the inner tube 210. Since portions of the inner tube 210 that have the supply lumens 212 formed therein are hollow, the portions each tend to easily have a decreased resistance to bending. However, employment of the above configuration leads to arrangement of the supply lumens 212 in the circumferential direction of the inner tube 210 as shown in FIG. 7. Consequently, the portions each of which easily has a decreased resistance to bending are present in a scattered manner in the circumferential direction of the inner tube 210. By doing so, retention of the resistance to bending can be facilitated regardless of the bending direction of the inner tube 210. Although not shown, it may be also preferable, for the same reason as the reason described above, that, in the cross section perpendicular to the longitudinal direction x of the outer tube 10, each of the centroids 212c of the plurality of supply lumens 212 is located on the one imaginary circle 210s having a center at the centroid 210c of the inner tube 210.

As shown in FIG. 7, it may be preferable that, in the cross section perpendicular to the longitudinal direction x of the outer tube 10, the plurality of supply lumens 212 are located on an imaginary circle 211s having a center at the centroid 211c of the guide wire lumen 211. In FIG. 7, the imaginary circle 211s having a center at the centroid 211c of the guide wire lumen 211 is indicated by an alternate long and two short dashes line. It may be preferable that, in the cross section perpendicular to the longitudinal direction x of the outer tube 10, all of the plurality of supply lumens 212 formed in the inner tube 210 are located on the imaginary circle 211s having a center at the centroid 211c of the guide wire lumen 211. Since portions of the inner tube 210 that have the supply lumens 212 formed therein are hollow, the portions each tend to easily have a decreased resistance to bending. However, employment of the above configuration leads to arrangement of the supply lumens 212 around the guide wire lumen 211 so that the supply lumens 212 are easily arranged in the circumferential direction of the inner tube 210, as shown in FIG. 7. Consequently, the portions each of which easily has a decreased resistance to bending are present in a scattered manner in the circumferential direction of the inner tube 210. By doing so, retention of the resistance to bending can be facilitated regardless of the bending direction of the inner tube 210. Although not shown, it may be also preferable, for the same reason as the reason described above, that, in the cross section perpendicular to the longitudinal direction x of the outer tube 10, each of the centroids 212c of the plurality of supply lumens 212 is located on one such imaginary circle 211s having a center at the centroid 211c of the guide wire lumen 211.

The fluid used for the above catheter 1 may be a liquid or a gaseous body. In a case where the fluid is a liquid, nitrogen or fluorocarbon may be used as the fluid. In a case where the fluid is a gaseous body, gas may be used as the fluid. Examples of the gas include argon, carbon dioxide, and nitrous oxide. The fluid used for the catheter 1 may be gas.

It may be preferable that, in the cross section perpendicular to the longitudinal direction x of the outer tube 10, a maximum width of each of the supply lumens 212 is smaller than a maximum width of the guide wire lumen 211. Consequently, it is possible to facilitate suppression of decrease, in the rigidity of the inner tube 210, that occurs owing to formation of the plurality of supply lumens 212 in the inner tube 210. It is also allowed to employ a configuration in which, in the cross section perpendicular to the longitudinal direction x of the outer tube 10, the maximum width of the supply lumen 212 is larger than the maximum width of the guide wire lumen 211, or the maximum width of the supply lumen 212 is equal to the maximum width of the guide wire lumen 211.

As shown in FIG. 9, in the cross section perpendicular to the longitudinal direction x of the outer tube 10, the inner tube 210 may have: a first supply lumen 2121; a second supply lumen 2122 formed to be adjacent to the first supply lumen 2121 in the circumferential direction of the inner tube 210; and a third supply lumen 2123 formed to be adjacent to the first supply lumen 2121 and the second supply lumen 2122 in the circumferential direction of the inner tube 210. In addition, the inner tube 210 may further have, in addition to the first supply lumen 2121, the second supply lumen 2122, and the third supply lumen 2123, one or more supply lumens which allow the fluid to pass therethrough from the proximal side to the distal side of the inner tube 210. For example, as shown in FIG. 10, the inner tube 210 may have the first supply lumen 2121, the second supply lumen 2122, the third supply lumen 2123, and a fourth supply lumen 2124. In the present description, one of or a combination of two or more of the first supply lumen 2121, the second supply lumen 2122, the third supply lumen 2123, and the fourth and larger-ordinal-number supply lumens which allow the fluid to pass therethrough from the proximal side to the distal side of the inner tube 210 are sometimes referred to as a supply lumen 212 or a plurality of supply lumens 212. As described above, employment of a configuration provided with the three or more supply lumens 212 makes it possible to, even when two of the supply lumens 212 are closed owing to bending of the catheter 1 which is passing through a bent body cavity, send the fluid through the remaining supply lumen(s) 212. Consequently, the fluid can be stably sent from the proximal side to the distal side.

As shown in FIG. 8, it may be preferable that: in the cross section perpendicular to the longitudinal direction x of the outer tube 10, the inner tube 210 has the first supply lumen 2121 and the second supply lumen 2122 formed to be adjacent to the first supply lumen 2121 in the circumferential direction of the inner tube 210; and, in the cross section perpendicular to the longitudinal direction x of the outer tube 10, an angle a formed between a half-line that has one end at the centroid 210c of the inner tube 210 and that passes through a centroid 2121c of the first supply lumen 2121 and a half-line that has one end at the centroid 210c of the inner tube 210 and that passes through the centroid 2122c of the second supply lumen 2122 is 105 degrees or larger and 135 degrees or smaller. In FIG. 8, the half-line having one end at the centroid 210c of the inner tube 210 and passing through the centroid 2121c of the first supply lumen 2121 and the half-line having one end at the centroid 210c of the inner tube 210 and passing through the centroid 2122c of the second supply lumen 2122 are indicated by alternate long and two short dashes lines.

The above angle a may be 105 degrees or larger, 110 degrees or larger, or 115 degrees or larger. The above angle a may be 135 degrees or smaller, 130 degrees or smaller, or 125 degrees or smaller. The above angle a may be 120 degrees.

As shown in FIG. 3, it may be preferable that the discharge flow path 31 is, in the cross section perpendicular to the longitudinal direction x of the outer tube 10, present so as to cover the entirety of the outer surface 213 of the inner tube 210. Although not shown, the discharge flow path 31 may be, in the cross section perpendicular to the longitudinal direction x of the outer tube 10, present so as to cover only a part of the outer surface 213 of the inner tube 210.

As shown in FIG. 1, FIG. 2, FIG. 5, and FIG. 6, the catheter 1 may further include a distal tip 60 in which a lumen 63 extending in the longitudinal direction x is formed. As shown in FIG. 1 and FIG. 2, a distal end portion of the outer tube 10 and a distal end portion of the inner tube 210 may be fixed to a proximal end portion of the distal tip 60.

The distal side of the discharge flow path 31 of the catheter 1 may be closed by fixing the distal end portion of the outer tube 10 and the distal end portion of the inner tube 210. A configuration in which the distal side of the discharge flow path 31 in the longitudinal direction x of the outer tube 10 is closed can be obtained by, for example, fixing the distal end portion of the outer tube 10 and the distal end portion of the inner tube 210 via the distal tip 60 as shown in FIG. 1 and FIG. 2. Although not shown, the distal end portion of the outer tube 10 and the distal end portion of the inner tube 210 may be welded and fixed to each other so as to close the distal side of the discharge flow path 31 without providing the distal tip 60. As shown in FIG. 5 and FIG. 6, in a case where the outer tube 10 has the balloon 50, the configuration in which the distal side of the discharge flow path 31 of the catheter 1 is closed can be obtained by fixing a distal end portion of the balloon 50 and the distal end portion of the inner tube 210. Consequently, a main direction in which the fluid jetted from the holes 40 to the outer side in the radial direction of the outer tube 10 moves can be set to be a direction toward the proximal side of the discharge flow path 31.

As shown in FIG. 1, FIG. 2, FIG. 5, and FIG. 6, it may be preferable that: the catheter 1 further includes a distal tip 60 in which a lumen 63 extending in the longitudinal direction x of the outer tube 10 is formed and which has an outer diameter decreasing from the proximal side toward the distal side; and the distal end portion of the outer tube 10 and the distal end portion of the inner tube 210 are fixed to a proximal end portion of the distal tip 60. By providing the distal tip 60 having an outer diameter decreasing from the proximal side toward the distal side, insertion of a distal end portion of the catheter 1 into a body cavity can be facilitated.

The shape of the distal tip 60 may be, for example, the shape of a hollow column, a hollow polygonal prism, a hollow truncated cone, or the like and may be the shape of a hollow truncated cone as shown in FIG. 1 and FIG. 2.

As a material forming the distal tip 60, a synthetic resin, a metal, or the like which are the same as those for the outer tube 10 may be used. The material forming the distal tip 60 and the material forming the outer tube 10 may be identical to or different from each other.

As shown in FIG. 1, FIG. 2, FIG. 5, and FIG. 6, a configuration may be employed in which a first handle 75 to be gripped by a user is connected to the proximal portion of the outer tube 10. In FIG. 2 and FIG. 6, the first handle 75 has a hollow portion extending in the longitudinal direction x of the outer tube 10. The shape of the first handle 75 may be, for example, a tubular shape. In FIG. 1, FIG. 2, FIG. 5 and FIG. 6, the outer tube 10 and the inner tube 210 are inserted into the hollow portion of the first handle 75.

As shown in FIG. 1, FIG. 2, FIG. 5, and FIG. 6, a configuration may be employed in which a second handle 76 is connected to the proximal portion of the inner tube 210. In FIG. 2 and FIG. 6, the second handle 76 has a hollow portion extending in the longitudinal direction x of the outer tube 10. The shape of the second handle 76 may be, for example, a tubular shape. In FIG. 2 and FIG. 6, the inner tube 210 is inserted into the hollow portion of the second handle 76. The second handle 76 may have a guide wire port as an opening into which a guide wire is inserted. Also, the second handle 76 may be connected to a fluid supply device 80 described later. As shown in FIG. 2, the proximal end of the inner tube 210 and the fluid supply device 80 may be directly connected to each other such that the fluid can be supplied to the supply lumens 212 of the inner tube 210.

Materials forming the first handle 75 and the second handle 76 are not particularly limited, and, for example, a synthetic resin such as a polyolefin resin such as polypropylene (PP) or polyethylene (PE), a polyester resin such as polyethylene terephthalate (PET), a polycarbonate resin, an ABS resin, or a polyurethane resin may be used.

A coating may be provided on the outer surface 13 of the outer tube 10. The coating may be provided on only a part of the outer surface 13 of the outer tube 10 or on the entirety of the outer surface 13 of the outer tube 10.

The coating to be provided on the outer surface 13 of the outer tube 10 may be a hydrophilic coating or a hydrophobic coating and may be selected according to the purpose. The coating can be provided on the outer surface 13 of the outer tube 10 by: immersing the outer tube 10 in a hydrophilic coating agent or a hydrophobic coating agent; applying the hydrophilic coating agent or the hydrophobic coating agent onto the outer surface 13 of the outer tube 10; or coating the outer surface 13 of the outer tube 10 with the hydrophilic coating agent or the hydrophobic coating agent. The coating agent may contain a drug and an additive.

Examples of the hydrophilic coating agent include: hydrophilic polymers such as polyvinyl alcohol, polyethylene glycol, polyacrylamide, polyvinylpyrrolidone, and methyl vinyl ether-maleic anhydride copolymers; hydrophilic coating agents made from a combination of any of these hydrophilic polymers; and the like.

Examples of the hydrophobic coating agent include polytetrafluoroethylene (PTFE), fluorinated ethylene propylene (FEP), perfluoroalkoxy alkanes (PFA), silicone oil, hydrophobic urethane resins, carbon coatings, diamond coatings, diamond-like carbon (DLC) coatings, ceramic coatings, substances each having an alkyl group or a perfluoro alkyl group at a terminal thereof and each having a low surface free energy, and the like.

The above coating may be provided also on an outer surface 61 of the distal tip 60. The coating may be provided on only a part of the outer surface 61 of the distal tip 60 or on the entirety of the outer surface 61 of the distal tip 60.

A cryoablation catheter system according to one or more embodiments of the present invention has the following gist. That is, the cryoablation catheter system includes the above catheter 1 and a fluid supply device 80 which supplies the fluid to the supply lumens 212, and the inner tube 210 is connected to the fluid supply device 80.

The fluid supply device 80 is not particularly limited as long as the fluid supply device 80 can supply the fluid to the inner tube 210. Examples of the fluid supply device 80 include a regulator, a flow rate controller, a pump, or the like connected to a fluid-storing container.

The inner tube 210 may be directly connected to the fluid supply device 80. For example, in the case of the catheter 1 shown in FIG. 2, FIG. 3, and FIG. 4, the three supply lumens 212 may converge into one lumen on the proximal side, and the proximal end of the inner tube 210 may be directly connected to the fluid supply device 80.

The inner tube 210 may be indirectly connected to the fluid supply device 80. For example, as shown in FIG. 6, a proximal end portion of the inner tube 210 may be connected to a distal end portion of a tubular member 77, and a proximal end portion of the tubular member 77 may be connected to the fluid supply device 80.

This application claims the benefit of the priority date of Japanese patent application No. 2022-179429 filed on Nov. 9, 2022. All of the contents of the Japanese patent application No. 2022-179429 filed on Nov. 9, 2022 are incorporated by reference herein.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

### REFERENCE SIGNS LIST

1: cryoablation catheter
10: outer tube
11: distal end of the outer tube
12: proximal end of the outer tube
13: outer surface of the outer tube
14: inner surface of the outer tube
15: inner cavity of the outer tube
16: tubular member
210: inner tube
210c: centroid of the inner tube
210s: imaginary circle having a center at the centroid of the inner tube
211: guide wire lumen
211c: centroid of the guide wire lumen
211s: imaginary circle having a center at the centroid of the guide wire lumen
212: supply lumen
212c: centroid of the supply lumen
2121: first supply lumen
2121c: centroid of the first supply lumen
2122: second supply lumen
2122c: centroid of the second supply lumen
2123: third supply lumen
2123c: centroid of the third supply lumen
2124: fourth supply lumen
2124c: centroid of the fourth supply lumen
213: outer surface of the inner tube
214: inner surface the inner tube
217: vertex portion
31: discharge flow path
40: hole
50: balloon
51: straight tube portion
52: distal-side tapered portion
53: proximal-side tapered portion
54: distal-side sleeve portion
55: proximal-side sleeve portion
60: distal tip
61: outer surface of the distal tip
62: inner surface of the distal tip
63: lumen of the distal tip
70: radiopaque marker
75: first handle
76: second handle
77: tubular member
80: fluid supply device

1. A cryoablation catheter comprising:
an outer tube having a distal end and a proximal end and extending in a longitudinal direction;
an inner tube extending in the longitudinal direction and disposed in an inner cavity of the outer tube; and
a discharge flow path, wherein:
in a cross section perpendicular to the longitudinal direction, an external shape of the inner tube is an irregular shape;
the inner tube comprises a guide wire lumen and a plurality of supply lumens, wherein, in the cross section perpendicular to the longitudinal direction, the plurality of supply lumens are formed in regions different from a region in which the guide wire lumen is formed, wherein:
the guide wire lumen is configured to have a guide wire inserted; and
the plurality of supply lumens are configured to allow a fluid to pass therethrough from a proximal side to a distal side of the inner tube;
the discharge flow path is located between an inner surface of the outer tube and an outer surface of the inner tube and is configured to allow the fluid to pass therethrough from a distal side to a proximal side of the outer tube; and
the inner tube comprises a hole located in a distal portion of the inner tube, wherein any of the supply lumens and the discharge flow path are in communication with each other through the hole.

2. The cryoablation catheter according to claim 1, wherein, in the cross section perpendicular to the longitudinal direction, the external shape of the inner tube is a polygonal shape.

3. The cryoablation catheter according to claim 1, wherein, in the cross section perpendicular to the longitudinal direction, the external shape of the inner tube is a triangular shape.

4. The cryoablation catheter according to claim 1, wherein:
in the cross section perpendicular to the longitudinal direction, the inner tube has a vertex portion protruding outward in a radial direction of the outer tube; and
the hole is formed in the vertex portion.

5. The cryoablation catheter according to claim 1, wherein:
in the cross section perpendicular to the longitudinal direction, a roundness of an external shape of the outer tube obtained according to the following formula is higher than a roundness of the external shape of the inner tube obtained according to the following formula:
(Rmax−Rmin)/Rmin, where:
R max represents a radius of a circumscribed circle, and R min represents a radius of an inscribed circle.

6. The cryoablation catheter according to claim 1, wherein, in the cross section perpendicular to the longitudinal direction, the guide wire lumen is formed at a position that overlaps with a centroid of the inner tube, and each of the supply lumens is formed at a position that does not overlap with the centroid of the inner tube.

7. The cryoablation catheter according to claim 1, wherein, in the cross section perpendicular to the longitudinal direction, the plurality of supply lumens are located on an imaginary circle having a center at a centroid of the guide wire lumen.

8. The cryoablation catheter according to claim 1, wherein, in the cross section perpendicular to the longitudinal direction, a maximum width of each of the supply lumens is smaller than a maximum width of the guide wire lumen.

9. The cryoablation catheter according to claim 1, wherein the outer tube comprises, on a distal portion thereof, a balloon which is expandable and shrinkable in a radial direction of the outer tube.

10. The cryoablation catheter according to claim 1, further comprising a distal tip comprising a lumen extending in the longitudinal direction, wherein: the distal tip has an outer diameter decreasing from a proximal side toward a distal side; and
a distal end portion of the outer tube and a distal end portion of the inner tube are fixed to a proximal end portion of the distal tip.

11. A cryoablation catheter system comprising:
the cryoablation catheter according to claim 1; and
a fluid supply device configured to supply the fluid to the supply lumens, wherein the inner tube is connected to the fluid supply device.
